(19) United States
(12) Patent Application Publication
(10) Pub. No.: US 2015/0203810 A1
(43) Pub. Date: Jul. 23, 2015

(54) MATERIALS AND METHODS FOR PROCESSING CELL POPULATIONS

(71) Applicant: Celula, Inc., San Diego, CA (US)

(72) Inventors: Andrew E. Senyei, La Jolla, CA (US); Haichuan Zhang, San Diego, CA (US); Kerry B. Gunning, San Diego, CA (US); Yi Zhang, San Diego, CA (US); Casey Walsh, Carlsbad, CA (US)

(21) Appl. No.: 14/408,549

(22) PCT Filed: Dec. 27, 2013
(86) PCT No.: PCT/US2013/046641

§ 371 Date: Dec. 16, 2014

Related U.S. Application Data
(60) Provisional application No. 61/662,246, filed on Jun. 20, 2012.

(51) Int. Cl. C12N 5/00; G01N 33/574; G01N 33/569

(52) U.S. Cl. CPC C12N 5/0068; G01N 33/56966; G01N 33/57492; C12N 2533/30

(57) ABSTRACT

Materials and methods for processing a cell population, such as enriching for a cell of interest from a cell population, are disclosed. The method may include: obtaining a cell population dispersed in a cytocompatible matrix; applying one or more labels to the cell population to distinguish a cell of interest from one or more cells of the cell population; identifying a portion of the cytocompatible matrix containing the cell of interest; and optionally isolating the portion of the cytocompatible matrix containing the cell of interest. The methods may, for example, advantageously provide reduced cell damage or loss relative to other procedures. Also disclosed are devices and compositions that may be useful for performing the methods.

### RELATED APPLICATIONS

This application claims priority to U.S. Provisional Patent Application 61/662,246, filed on Jun. 20, 2012, titled “MATERIALS AND METHODS FOR PROCESSING CELL POPULATIONS,” which is incorporated herein by reference in its entirety where permitted.

### BACKGROUND

Numerous materials and methods exist for enriching a cell of interest from a heterogeneous cell population. For example, fluorescence-activated cell sorting (FACS) is a specialized type of flow cytometry where a narrow flow of individual cells can be sorted based on light scattering or fluorescent characteristics. As another example, microfluidic devices have been used to enrich cells. These existing techniques may exhibit significant cell damage or loss due to, for example, cell adhesion to surfaces during processing. This cell loss can be particularly undesirable when enriching rare cells from a heterogeneous cell population. Thus, there exists a need for improved materials and methods for enriching a heterogeneous mixture of cells for a cell of interest.

### SUMMARY

Some embodiments disclosed herein include a method. The method can include obtaining a cell population dispersed in a cytocompatible matrix; applying a label to the cell population to distinguish a cell of interest from one or more cells of the cell population; and identifying a portion of the cytocompatible matrix containing the cell of interest.

In some embodiments, the method further includes isolating the portion of the cytocompatible matrix containing the cell of interest.

In some embodiments, the cytocompatible matrix includes a polymer. In some embodiments, the cytocompatible matrix includes a hydrogel.

In some embodiments, the cell population is heterogeneous. In some embodiments, the cell population is obtained from a blood sample, in some embodiments the blood sample is from a pregnant woman. In some embodiments, the cell population includes maternal cells and fetal cells. In some embodiments, the cell of interest is a fetal cell. In some embodiments, the cell of interest is a cancer cell. In some embodiments, the cell of interest is a stem cell.

In some embodiments, obtaining the cell population dispersed in a cytocompatible matrix includes combining the cell population with a composition comprising a polymer and crosslinking the polymer to form the cytocompatible matrix. In some embodiments, crosslinking the polymer to form the cytocompatible matrix includes applying radiation to the polymer, heating the polymer, adjusting a pH of the composition, or combining a crosslinking agent with the composition.

In some embodiments, applying the labels to the cell population includes combining the label with the cell population before the cell population is dispersed in the cytocompatible matrix. In some embodiments, applying the label to the cell population includes combining the label with the cell population after crosslinking the polymer.

In some embodiments, obtaining the cell population dispersed in the cytocompatible matrix includes combining the cell population with a composition including a monomer and polymerizing the monomer to form the cytocompatible matrix.

In some embodiments, the cytocompatible matrix includes a polymer selected from the group consisting of poly(alkylene oxide), a starch, a cellulose, a polysaccharide, polyurethane, polyvinyl alcohol, polyvinyl ether, polyacrylate, polyvinylpyrolidone, polyesters, polyacrylamide, polyglycolic acid, polylactic acid, a protein, copolymers thereof and derivatives thereof.

In some embodiments, the cytocompatible matrix is light-transmissive. In some embodiments, the cytocompatible matrix has a light transmittance of at least about 50% for visible light. In some embodiments, the cytocompatible matrix has a viscosity of at least about 50 cP. In some embodiments, the cytocompatible matrix is porous. In some embodiments, the cytocompatible matrix is photodegradable or enzymatically degradable. In some embodiments, the cytocompatible matrix has a mass swelling ratio at approximately equilibrium conditions of less than about 500.

In some embodiments, applying one or more labels to the cell population comprises applying the labels to a surface of the cytocompatible matrix containing the cell population. In some embodiments, at least one of the labels identifies a cellular surface marker or an intracellular marker or structure. In some embodiments, the intracellular marker is a nucleic acid marker or a protein marker. In some embodiments, at least one of the labels is an antibody. In some embodiments, the label is a stain, preferably to identify a cellular structure. In some embodiments the stain is a nuclei stain, for example, hematoxylin, neutral/toluylene red, or Nile blue.

In some embodiments, the method includes applying at least two labels to the cell population. In some embodiments, a first label and a second label are applied to the cell population, the first label is configured to identify a cellular surface marker, and the second label is configured to identify an intracellular marker or structure.

In some embodiments, at least one of the labels is fluorescent-labeled or radio-labeled. In some embodiments, at least one of the labels comprises a magnetic or paramagnetic particle.

In some embodiments, at least one of the labels identifies a marker for fetal cells. In some embodiments, the marker for fetal cells is selected from the group consisting of CD71, CD34, CD45, and CD235a.

In some embodiments, at least one of the labels identifies a marker for a maternal cell. In some embodiments, the marker for the maternal cell is selected from the group consisting of CD2, CD3, CD11b, CD14, CD15, CD16, CD19, CD56, CD123, and CD61.

In some embodiments, at least one of the labels identifies a marker for a cancer cell.

In some embodiments, identifying a portion of the cytocompatible matrix containing a cell of interest from the cell population includes detecting radiation corresponding to at least one of the labels.

In some embodiments, identifying a portion of the cytocompatible matrix containing a cell of interest from the cell population includes detecting a magnetic field or a magnetic force corresponding to at least one of the labels.

In some embodiments, isolating the portion of the cytocompatible matrix containing the cell of interest includes mechanically separating the portion of the cytocompatible matrix containing the cell of interest from the cytocompatible matrix.

In some embodiments, isolating the portion of the cytocompatible matrix containing the cell of interest includes selectively degrading the cytocompatible matrix in the portions of the cytocompatible matrix containing the cell of interest and removing the degraded portions from the cytocompatible matrix.

In some embodiments, isolating the portion of the cytocompatible matrix containing the cell of interest includes selectively degrading the cytocompatible matrix adjacent to the portion containing the cell of interest and separating the degraded portions from the portion containing the cell of interest.

Some embodiments disclosed herein include a device having a substrate; a layer disposed on the substrate, wherein the layer comprises a cytocompatible matrix, and the layer has a thickness of about 2 mm or less; and a cell population dispersed within the layer.

Some embodiments disclosed herein include a composition including a cytocompatible matrix and one or more labels to distinguish a cell of interest from one or more cells of the cell population.

Some embodiments disclosed herein include a composition including a cytocompatible matrix, a cell population, and one or more labels to distinguish a cell of interest from one or more cells of the cell population. In some embodiments, the cell population includes maternal cells and fetal cells.

### BRIEF DESCRIPTION OF THE DRAWINGS

FIG. 1 is a flow diagram that depicts some embodiments of a method for processing a cell population using a cytocompatible matrix.

FIG. 2 is a perspective view of one example of a cell population dispersed in a cytocompatible matrix.

FIG. 3 shows a cell population dispersed in a cytocompatible matrix according to the procedure in Example 1 using bright field microscopy.

FIG. 4 shows a stained cell population dispersed in a cytocompatible matrix according to the procedure in Example 1 using fluorescence microscopy.

FIG. 5 shows a contrast-enhanced image of a target cell identified using epsilon and gamma hemoglobin antibodies according to the procedure in Example 1.

### DETAILED DESCRIPTION

Disclosed herein are materials and methods for processing a cell population using a cytocompatible matrix. In some embodiments, the method can is used for enriching for a cell of interest from a heterogeneous cell population. The method may include: obtaining a cell population dispersed in a cytocompatible matrix; applying one or more labels to the cell population to distinguish a cell of interest from one or more cells of the cell population; and identifying a portion of the cytocompatible matrix containing the cell of interest. In some embodiments, the method may further include isolating the portion of the cytocompatible matrix containing the cell of interest. The methods may, for example, advantageously provide reduced cell damage or loss relative to other procedures (e.g., other cell enrichment procedures). Also disclosed herein are devices and composition for performing the methods disclosed in the present application.

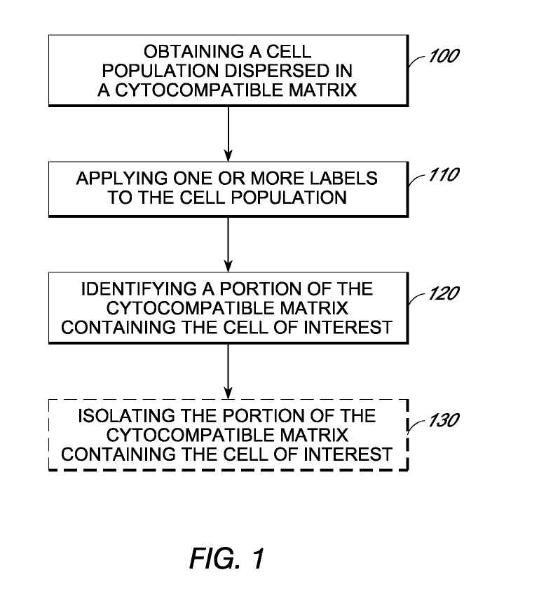

FIG. 1 is a flow diagram that depicts some embodiments of a method for processing a cell population using a cytocompatible matrix. In some embodiments, the method can perform enriching for a cell of interest from a cell population. The method may include: an operation “Obtaining a cell population dispersed in a cytocompatible matrix,” illustrated in block 100; an operation “Applying one or more labels to the cell population to distinguish a cell of interest from one or more cells of the cell population,” illustrated in block 110; an operation “Identifying a portion of the cytocompatible matrix containing the cell of interest,” illustrated in block 120; and an optional operation “Isolating the portion of the cytocompatible matrix containing the cell of interest,” illustrated in block 130. Although operations 100, 110, 120, and 130 may be performed sequentially, it will be appreciated that one or more of these operations may be performed at about the same time. These operations may also be performed in a different order than is depicted in FIG. 1.

At operation 100 “Obtaining a cell population dispersed in a cytocompatible matrix,” a cell population is obtained for processing. The cell population dispersed in a cytocompatible matrix may, in some embodiments, be provided by a third-party. As an example, a doctor may obtain a sample from a patient, disperse the cells in a cytocompatible matrix, and then send the sample to a diagnostic testing facility for further processing.

The cell population is not particularly limited, and may be, for example, a cell population in which enrichment is desired. The cell population may include prokaryotic cells, eukaryotic cells, or a combination of these cells. The cell population, in some embodiments, can be mammalian (e.g., from a human). In some embodiments, the cell population is obtained from a body fluid of a mammal, such as blood, amniotic fluid, and the like. The cell population may optionally be enriched or pre-processed before dispersion in the cytocompatible matrix. For example, cells may be subject to debulking using density gradient centrifugation over a density gradient medium, such as PERCOLL (Sigma-Aldrich, St. Louis, Mo.) before dispersing in the cytocompatible matrix. The cell population may be heterogeneous or homogeneous. That is, the cell population may include two or more different cell types (e.g., two, three, four, or more different cell types), or the cell population may have only a single cell type.

The cell population may, in some embodiments, include maternal cells and fetal cells. For example, the cell population may be obtained from the blood sample of a pregnant female that contains maternal cells and fetal cells. In some embodiments, the cell population includes a cancer cell. For example, the cell population may be obtained from a cancerous tissue sample of a subject.

The cell population can be dispersed in various types of cytocompatible matrices. The cytocompatible matrix may be generally any inert material that is cytocompatible. The cytocompatible matrix is therefore different from fixatives typically used in histology to preserve biological tissues. Numerous cytocompatible matrices are known in the art and commonly used in various tissue engineering and drug delivery applications. In some embodiments, the cytocompatible matrix may be configured so that the genome within cells of the cell population remains intact. In some embodiments, the cytocompatible matrix includes a polymer. In some embodiments, the cytocompatible matrix is a hydrogel.

Numerous polymers are known in the art for forming a cytocompatible matrix that can be generally non-toxic and/or inert. Non-limiting examples of suitable polymers include poly(alkylene oxide), starches, celluloses, polysaccharides, polyurethane, polyvinyl alcohol, polyvinyl ether, polyacrylate, polyacrylamide, polyvinylpyrolidone, polyglycolic acid, polylactic acid, and proteins. Some specific examples of polymers include polyethylene glycol, polypropylene glycol, carboxy methyl starch, hyaluronic acid, chitosan, alginic acid, polyacrylic acid, gelatin, collagen, and fibrin. These polymers may be used either alone or in combination to form the cytocompatible matrix. Various copolymers of these polymers, including graft copolymers, may also be used. For example, a copolymer of polyethylene glycol and polyacrylate may be used, such as polyethylene glycol diacrylate.

The cytocompatible matrix may be light-transmissive to permit viewing the cell population and/or labels (e.g., labels applied to the cell population in operation 110 discussed further below). In some embodiments, the cytocompatible matrix is configured to be light-transmissive so that the cell population may be viewed (e.g., using a standard microscope). In some embodiments, the cytocompatible matrix is configured to be light-transmissive so that light emitted from one or more labels can be detected. For example, the cytocompatible matrix may be light-transmissive so that fluorescent light emitted from a fluorescent-tagged antibody can be detected. The cytocompatible matrix may, for example, have a light transmittance for visible light that is, or is at least, or at least about 30%, 35%, 40%, 45%, 50%, 55%, 60%, 65%, 70%, 75%, 80%, 85%, 90%, 95% 98%, or 100%, or a range defined by any two of the preceding values. In some embodiments, the transmittance for visible light is between 50% and 100%, or between 75% and 95%. In some embodiments, the cytocompatible matrix is generally transparent. The skilled artisan, guided by the teachings of the present application, will appreciate that the light-transmissive properties of the cytocompatible matrix can be readily modified by adjusting, for example, the type of polymer, concentration of polymer, degree of crosslinking, molecular weight, and other characteristics of the cytocompatible matrix.

The viscosity of the cytocompatible matrix may, for example, have a viscosity that is, or is at least, or at least about 1 cP, 20 cP, 50 cP, 100 cP, 500 cP, 1,000 cP, 2,000 cP, 5,000 cP, or 10,000 cP. The cytocompatible matrix may, for example, have a viscosity that is, or is no more than, or no more than about 100,000 cP, 50,000 cP, 20,000 cP; or 10,000 cP. In some embodiments, the cytocompatible matrix has a viscosity of between 1 cP and 100,000 cP, or between 20 cP and 20,000 cP, or between 1,000 cP and 10,000 cP.

The swelling properties of the cytocompatible matrix may have a mass swelling ratio (weight ratio of solids to water in the cytocompatible matrix at near equilibrium conditions), for example, that is, or is no more than, or is no more than about 500, 250, 100, 50, 25, or 10. The cytocompatible matrix may have a mass swelling ratio, for example, that is, or is at least, or is at least about at least about 1, 5, 10, 20, or 50. In some embodiments, the cytocompatible matrix has a mass swelling ratio from about 1 to about 500.

The weight average molecular weight of any polymer in the cytocompatible matrix (before crosslinking) can be modified to adjust various properties of the cytocompatible matrix (e.g., viscosity). The weight average molecular weight of the polymer, for example, is, or is at least, or is at least about 500 Da, 1,000 Da, 5,000 Da, 10,000 Da, 50,000 Da; or 100,000 Da. The weight average molecular weight of the polymer, for example, is, is no more than, or is no more than about 1 million Da, 500,000 Da, 250,000 Da, 100,000 Da, 50,000 Da or 10,000 Da. In some embodiments, the weight average molecular weight of the polymer can be between 500 Da and 1 million Da, or between 1,000 Da and 500,000 Da, or between 5,000 Da and 250,000 Da.

The amount of optional polymer can be modified to adjust various properties of the cytocompatible matrix (e.g., swelling). The amount of polymer in the cytocompatible matrix, for example, is, or is at least, or is at least about 0.1% by weight, 0.5% by weight, 1% by weight, 2% by weight; or 5% by weight. The amount of polymer in the cytocompatible matrix, for example, is, or is no more than, or is no more than about 20% by weight, 15% by weight, 10% by weight; or 5% by weight. In some embodiments, the amount of polymer in the cytocompatible matrix can be between 0.1% and 20% by weight, or between 0.5% and 15% by weight, or between 1% and 10% by weight.

The cytocompatible matrix may include water as the main component. The amount of water in the cytocompatible matrix, for example, is, or is at least, or is at least about 50% by weight, 70% by weight, 90% by weight, or 95% by weight. The amount of water in the cytocompatible matrix, for example, is, or is no more than, or is no more than about 99.9%, 99%; or 95% by weight. In some embodiments, the amount of water in the cytocompatible matrix can be between 50% and 99.9% by weight, or between 70% and 99% by weight.

The cytocompatible matrix may also be configured to be permeable to one or more labels. As will be discussed further below, one or more labels can be applied to the cell population. If the labels are applied to the cell population while dispersed within the cytocompatible matrix, the cytocompatible matrix can have sufficient permeability for the label to transport (e.g., diffuse) through the cytocompatible matrix to reach targeted cell markers. For example, the cytocompatible matrix may have sufficient permeability for an antibody to transport within the cytocompatible matrix to bind to a cell surface marker. In some embodiments, the cytocompatible matrix can be porous to increase permeability. The skilled artisan, guided by the teachings of the present application, will appreciate that the permeability can be readily adjusted by modifying, for example, the concentration polymer, molecular weight, degree of crosslinking, and other factors to obtain sufficient permeability.

In some embodiments, the cytocompatible matrix may be degradable. As will be discussed further below, the cytocompatible matrix may be degraded to isolate a portion of the cytocompatible matrix (e.g., during operation 130 depicted in FIG. 1). The cytocompatible matrix can optionally be photodegradable. For example, photodegradable crosslinking agents can be combined with a polymer to form a photodegradable hydrogel. Kloxin, A. et al., Nature Protocols, Vol. 5(12), pp. 1867-87 (2010), which is incorporated herein by reference in its entirety, describes one example of a photodegradable polymer using o-nitrobenzylether-based crosslinking agents and polyethylene glycol. The cytocompatible matrix may optionally be enyzmatically degradable. This may be achieved, for example, using various polysaccharides and proteins that have known enzymes for degrading these polymers. As non-limiting examples, hyaluronic acid can be enzymatically degraded by hyaluronidase, while collagen can be enzymatically degraded by collagenase.

In some embodiments, obtaining a cell population dispersed in a cytocompatible matrix includes combining (e.g., mixing) the cell population with a composition having a polymer, and crosslinking the polymer to form the cytocompatible matrix. As an example, a body fluid (e.g., blood) or tissue (e.g., biopsy) can be mixed with a polymer and the polymer is then crosslinked to form a cytocompatible matrix having a cell population dispersed within a hydrogel. As noted above, the cell population can be pre-processed (e.g., debulked, tissue dissociation) before combining with the polymer and crosslinking In some embodiments, sterile, isotonic fluids (e.g., saline) may also be combined with the polymer and the cell population. In some embodiments, the cell population is diluted with a solution or buffer (e.g., saline or PBS) before combining with the cytocompatible matrix.

The method for crosslinking may vary, for example, depending on the polymer used. Any known method for crosslinking the polymer may be used so long as the crosslinking has low reactivity with the cell population. In other words, the crosslinking reaction does not significantly damage the cell population (e.g., the reaction is cytocompatible). In some embodiments, crosslinking the polymer includes applying radiation to the polymer, heating the polymer, adjusting a pH of the composition containing the polymer, or combining a crosslinking agent with the composition containing the polymer. For example, a thiol-modified hyaluronic acid may be combined with a vinyl-containing crosslinking agent to form the cytocompatible matrix. Horkay, F. et al., Polymer Vol. 51, (2010), pp. 4424-4430, incorporated by reference herein in its entirety, describes one example of crosslinking a thiol-modified hyaluronic acid with polyethylene glycol diacrylate.

In some embodiments, obtaining a cell population dispersed in a cytocompatible matrix includes combining (e.g., mixing) the cell population with a composition having a monomer, and polymerizing the monomer to form the cytocompatible matrix. Any known method for polymerizing the monomer may be used so long as the process has low reactivity with the cell population. The polymerization may, in some embodiments, include free-radical polymerization. For example, the monomer may be a vinyl-containing monomer, such as polyethylene glycol diacrylate, and radiation may be applied to polymerize the vinyl-containing monomer units. A polymerization initiator, such as the photoinitiator IRGACURE 2959, may also be combined with the monomer to initiate polymerization.

The number of cells in the cell population may vary significantly. The cell population may include, for example, at least about 10 cells; at least about 100 cells; at least about 1,000 cells; at least about 10,000 cells; at least about 100,000 cells; at least about 1,000,000 cells; at least about 5,000,000 cells; at least about 10,000,000 cells; or at least about 100,000,000 cells.

The density of cells within the hydrogel can be modified, for example, by varying the amount of hydrogel (or hydrogel precursor, such as a polymer or monomer composition described above) combined with the cell population. The density of cells within the hydrogel, for example, is, or is at least, or is at least about 1 thousand, 10 thousand, 100 thousand, 1 million, 10 million, 20 million, 50 million, 100 million, 1 billion, 10 billion, or 100 billion cells per mL. The density of cells within the hydrogel, for example, is no more than, or is no more than about 100 billion, 10 billion, 1 billion, 100 million, 50 million, 20 million, 10 million, 1 million, 100 thousand, or 10 thousand cells per mL. In some embodiments, the density of cells within the hydrogel is between 1 thousand and 100 billion cells per mL, or between 1 million and 100 million cells per mL, or between 10 million and 50 million cell per mL.

In some embodiments, a cell-containing solution (e.g., saline) can be combined with the hydrogel (or hydrogel precursor, such as a polymer or monomer composition described above). A volume of the cell-containing solution relative to a volume of the hydrogel can be between 1:100 to about 100:1, or between 1:10 and 1:1.

The dimensions of the cytocompatible matrix containing the cell population can vary. In some embodiments, the cytocompatible matrix can be a film. The film may have a thickness of, for example, less than about 2 mm; less than about 1 mm; less than about 500 μm; less than about 250 μm; less than about 100 μm; less than about 75 μm; or less than about 50 μm. The film may have a large surface area to aid viewing cells within the cytocompatible matrix. One side of the film may have a surface area of at least about 1 cm²; at least about 5 cm²; at least about 10 cm²; at least about 50 cm²; at least about 500 cm²; or at least about 1 m².

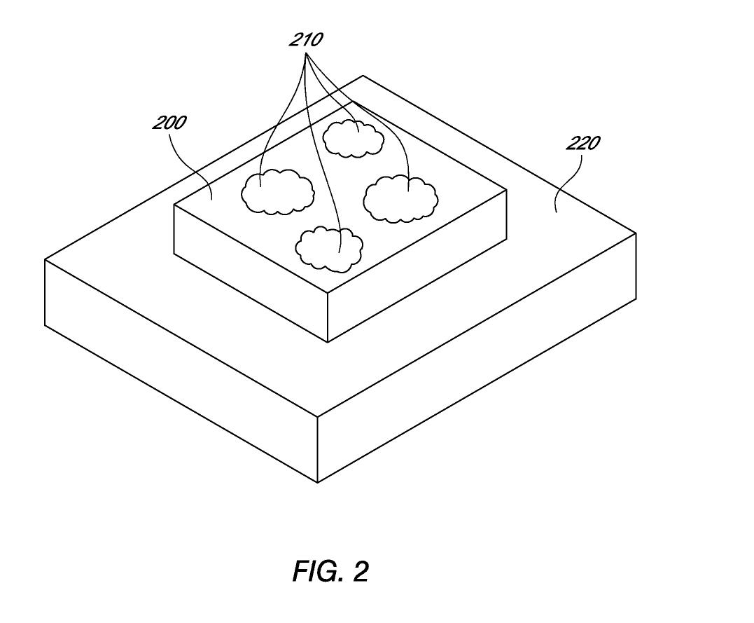

FIG. 2 is a perspective view of one example of a cell population dispersed in a cytocompatible matrix. Film 200 includes cell population 210 dispersed within a cytocompatible matrix. Film 200 can include any of the cytocompatible matrices and cell populations disclosed within the present application. Film 200 can also be configured so that cell population 210 is visible (e.g., using a standard microscope). Film 200 is disposed on substrate 220. In some embodiments, the substrate is transparent, or has a light transmittance for visible light as described above for the matrix. The cytocompatible matrix can be disposed, for example, on a glass slide, a polymer layer, or a hydrogel film (e.g., substrate 220 depicted in FIG. 2 may be a glass slide, a polymer layer, or a hydrogel film). The cytocompatible matrix may, in some embodiments, be disposed between two substrates. For example, the cytocompatible matrix can be sandwiched between two glass slides or two hydrogel films, which may be cell free hydrogel films. In some embodiments, the cytocompatible matrix can be disposed on a substrate that includes a regular pattern, such as a grid, to aid locating cells of interest in the matrix. As an example, substrate 230 can be a glass slide with grid markings. When a cell of interest is identified, the cell location can be recorded based on the grid markings and used when isolating portions of the cytocompatible matrix.

Returning to FIG. 1, at operation 110 “Applying one or more labels to the cell population to distinguish a cell of interest from one or more cells of the cell population,” a label can be applied to distinguish a cell of interest. The label applied may vary depending on the cell of interest. Generally, any label used in flow cytometry techniques can be applied to the cell population. The labels can be, for example, fluorescent-tagged monoclonal antibodies, radio-labeled monoclonal antibodies, or cell stains or dyes. In some embodiments, at least one of the labels includes a magnetic particle or paramagnetic particle which can be detected using a magnetic field. In some embodiments, at least one of the labels includes a nanoparticle, such as a quantum dot, which can be detected using fluorescence. However, the present application is not limited to labels used in flow cytometry.

In some embodiments, at least one of the labels is configured to identify a cellular surface marker (e.g., antigen). For example, the labels can include an antibody having a fluorescent tag that is configured to bind to a cell surface marker such as CD71. In some embodiments, at least one of the labels is configured to identify an intracellular marker (e.g., a cellular structure, protein marker or nucleic acid marker). As an example, at least one of the labels can include an anti-fetal hemoglobin antibody that is configured to bind fetal hemoglobin within a fetal cell. As another example, bisbenzimide may be applied to the cell population to identify cells containing DNA.

The one or more labels can be applied to the cell population using various methods. In some embodiments, the labels can be applied to a surface of the cytocompatible matrix containing the cell population. The labels may then transport (e.g., diffusion, electrophoresis, optophoresis, centrifugation, magnetic field, or electrical field) through the cytocompatible matrix to reach the cell population. In some embodiments, the labels can be applied to the cell population before the cell population is dispersed in the cytocompatible matrix. For example, the labels may be combined with the cell population and a polymer before crosslinking, or the labels may be combined with the cell population and a monomer before polymerization.

In some embodiments, only one label is applied to the cell population. For example, only an anti-fetal hemoglobin antibody is applied to the cell population to identify fetal cells. In some embodiments, a plurality of labels (e.g., two, three, four, or more labels) are applied to the cell population. In some embodiments, at least one label configured to identify a cellular surface marker is applied to the cell population, and at least one label configured to identify an intracellular marker is applied. For example, a radio-labeled antibody for CD71 and bisbenzimide is applied to the cell population. In some embodiments, at least two labels configured to identify different cellular surface markers are applied to the cell population. As an example, an antibody configured to bind to CD71 and an antibody configured to bind to CD123 are both applied to the cell population.

The labels may, in some embodiments, be selected for identifying fetal cells from a cell population including maternal cells and fetal cells. For example, identifying a fetal cell in a cell population obtained from a maternal blood sample. The labels may, for example, be configured to identify at least one marker for a fetal cell. Non-limiting examples of markers for a fetal cell include CD71, CD34, CD235a, CK19, Human leukocyte antigen (HLA) markers and fetal hemoglobin. The labels may, for example, be configured to identify at least one marker for non-fetal cells (e.g., maternal cells). Non-limiting examples of markers for non-fetal cells include CD2, CD3, CD11, CD14, CD15, CD16, CD19, CD45, CD56, CD66, CD123, and CD61. In some embodiments, at least one label for identifying a marker for a fetal cell is applied to the cell population, and at least one label for identifying a marker for a maternal cell is applied to the cell population.

The labels may, in some embodiments, be selected for identifying cancer cells. For example, identifying a cancer cell from a tissue sample, a fine needle aspirate sample, or a blood sample obtained from a subject. As a specific example, an anti-EGFR antibody may be applied to cells to identify certain cancer cells (e.g., breast cancer).

Various other cellular characteristics can be identified using the methods disclosed herein. The labels may be configured to identify cells that exhibit a specific response to external stimuli. Thus, in some embodiments, the method can optionally include applying a stimulus to the cell population and subsequently (or at about the same time) applying one or more labels to the cell population. The stimulus may be applying one or more molecules to the cell population, such as drugs (e.g., anti-cancer drugs), hormones, proteins, and the like. For example, an anti-cancer drug may be applied to a cell population and then a label configured to identify a cell surface marker can be applied to the cell population. Cells that respond to the anti-cancer drug by modulating expression of the cell surface marker may be identified using the label. In some embodiments, anti-cancer drugs are applied to a cancer cell population in the cytocompatible matrix to identify cancer cells that are sensitive or resistant to the anti-cancer drugs.

At operation 120 “Identifying a portion of the cytocompatible matrix containing the cell of interest,” a cell of interest can be identified using the one or more labels applied to the cell population. The method for locating a cell of interest can vary depending upon, for example, the type of cell that is of interest and the labels applied to the cell population.

In some embodiments, at least one fluorescent-tagged label (e.g., a dye or antibody) is applied to the cell population, and radiation can be applied to the matrix that is effective for the label to fluoresce. The portions of the matrix exhibiting fluorescence that corresponds to the label can be identified as containing the cell of interest if the label is configured to identify a marker for the cell of interest. Alternatively, the portions without fluorescence corresponding to the label can be identified as containing the cell of interest if the label is configured to identify a marker for a cell other than the cell of interest. The portions of the matrix may be identified, for example, using a fluorescence microscope or a suitable camera to observe the fluorescence. The label may, in some embodiments, have color detectable by the human eye. Thus, portions of the matrix may be identified by viewing the matrix under a microscope without applying radiation that produces fluorescence.

In some embodiments, at least one label can be radio-labeled, and radiation that corresponds to the label can be measured and correlated with portions of the matrix containing the cell of interest. In some embodiments, at least one label can include a magnetic particle or paramagnetic particle, and a magnetic field or magnetic force that corresponds to the label can be measured and correlated with portions of the matrix containing the cell of interest.

The skilled artisan, guided by the teachings of the present application, will appreciate that multiple labels may be used in combination to identify portions of the matrix having a greater probability of containing the cell of interest. The identification of portions in the matrix may also be optionally automated using an appropriate electronic device, such as a digital camera with image processing software.

The methods of the present application may be used to identify various cells of interest. In some embodiments, the cell of interest is abundant within the cell population. The number of cells of interest relative to a total number of cells in the cell population, for example, is, is at least, or at least about 10%, 20%, 30%, 40%, 50%, 60%, 70%, 80%, 90%, or 99%, or a ranged defined by any two of the preceding values. In other embodiments, the cell of interest may be rare within the cell population. The number of cells of interest relative to a total number of cells in the cell population, for example, is, or is no more than, or is no more than about 1%, 0.1%, 0.01%, 0.001%, or 0.0005%. For example, the cell of interest may be fetal cells obtained from a maternal blood sample. The estimated proportion of fetal cells is about 1-10 out of 1,000,000 cells.

At operation 130 “Isolating the portion of the cytocompatible matrix containing the cell of interest,” the portions of the matrix identified as containing the cell of interest can be optionally isolated. In some embodiments, isolating the portion of the cytocompatible matrix can include mechanically separating the portion of the cytocompatible matrix containing the cell of interest from the cytocompatible matrix. For example, the cytocompatible matrix can be cut into two or more parts using a scalpel, cutting die, or similar tools, and the portion of the cytocompatible matrix containing the cell of interest removed using standard techniques, such as by suction with a pipette or using tweezers.

The portion of the cytocompatible matrix containing the cell of interest may, in some embodiments, be isolated by selectively degrading the cytocompatible matrix in the portions containing the cell of interest. The degraded portion may then be separated from the cytocompatible matrix using standard techniques, such as by washing, suction or capillary action. As discussed above, the matrix may, for example, be composed of materials that are photodegradable or enzymatically degradable. Thus, in some embodiments, radiation effective to degrade the cytocompatible matrix can be selectively applied to the portion of the matrix identified as having the cell of interest. The photodegraded portion can then be removed. In some embodiments, an appropriate enzyme can be selectively applied (e.g., using a micropipette) to the portion of the matrix identified as having the cell of interest. The enzymatically degraded portions may then be removed.

The portion of the cytocompatible matrix containing the cell of interest may, in some embodiments, be isolated by selectively degrading at least regions of the matrix adjacent to the portion containing the cell of interest. For example, radiation effective to degrade the matrix can be applied to peripheral regions surrounding the portion containing the cell of interest. The non-degraded portion containing the cell of interest may then be separated using standard techniques, such by suction, washing, and the like. Similarly, enzymatic degradation may be used to selectively degrade at least regions of the matrix adjacent to the portion containing the cell of interest.

The isolated portion of the matrix can be a single continuous region of the matrix, or two or more distinct regions of the matrix containing the cell of interest (e.g., two, three, or more portions). Thus, for example, the operation of cutting and removing regions (or degrading and removing regions) may be repeated one or more times. If two or more distinct regions are isolated, they may all be placed into a single container or separate containers for further processing.

The size of each region removed from the matrix may be relatively small. The volume of each region removed from the matrix can be, for example, less than about 10 mm³; less than about 5 mm³; less than about 1 mm³; less than about 0.5 mm³; less than about 0.1 mm³; less than about 0.05 mm³; or less than about 0.01 mm³. The total size of the isolated portion of the matrix may also be relatively small. The total volume of the isolated portion of the matrix can be, for example, less than about 100 mm³; less than about 10 mm³; less than about 5 mm³; less than about 1 mm³; less than about 0.5 mm³; less than about 0.1 mm³; less than about 0.05 mm³; or less than about 0.01 mm³.

After isolating the portion of the matrix containing the cell of interest, the matrix may be subsequently degraded to provide access to the cells for further processing. For example, the portion containing the cell of interest can be enzymatically degraded or photodegraded. The cells of interest may then, for example, be cultured to increase the cell count. The methods disclosed in the present application may therefore be completed, in some embodiments, so that the cell of interest can be viable. The viable cells may provide an intact genome suitable for amplification. Accordingly, the processes disclosed in the present application may not include fixing the cells as typically used in histology (e.g., does not include fixation with an aldehyde or alcohol to preserve non-living cells).

The methods disclosed in the present application may have several advantages over existing procedures (e.g., cell enrichment procedures). In some embodiments, the process may have a limited number (if any) of centrifuging steps applied to the cell population. The method may include centrifuging the cell population, for example, no more than two times; no more than one time; or zero times. By reducing or eliminating centrifuging steps, this may reduce cell damage or loss. In some embodiments, the process can be completed quickly. The method may take, for example, less than 2 days; less than 36 hours, less than a day, less than 18 hours, less than 12 hours, less than 6 hours, less than 1 hour, or less than 5 minutes.

The methods of the present application may yield an enriched cell population for the cell of interest. The number of cells of interest relative to a total number of cells in the enriched cell population, for example, is, or is at least, or is at least about 0.001%, 0.01%, 0.1%, 1%, 2%, or 5%, 20%, or 50%, or a range defined by any two of the preceding values.

Various optional post-processing operations can be included with the methods disclosed herein. In some embodiments, the cell of interest within the isolated portion of the matrix can be subject to screening or genotyping. In some embodiments, the cells within the isolated portion of the matrix can be amplified. Non-limiting examples of amplification procedures include whole genome amplification, whole transcriptome amplification, targeted nucleic acid amplification, generation of a proxy for a nucleic acid sequence, or cell division. In some embodiments, DNA from the cells within the isolated portion of the matrix can be amplified using polymerase chain reaction (PCR).

The optional amplification operation may be performed on the isolated portion of the matrix without separating the cell from the cytocompatible matrix (or a degraded form of the cytocompatible matrix). As an example, the isolated portion of the matrix (e.g., the isolated portion of the matrix obtained from operation 130 depicted in FIG. 2) can be degraded and the degraded matrix including the cells can be directly used for amplification without separating the cells from the degraded matrix (e.g., without using centrifugation to isolate cells from the degraded matrix). Thus, in some embodiments, the cytocompatible matrix may be compatible with amplification techniques, such as PCR.

The methods disclosed herein may be used to enrich for fetal cells to perform screening or genotyping of the enriched cell population for a fetal identifier. Commonly assigned U.S. Publication No. 2011/0086769 discloses various techniques for detecting alleles, genomes, and transcriptomes in admixtures of two cell types from different individuals (e.g., fetal and maternal cells) and is hereby incorporated by reference in its entirety.

Some embodiments disclosed herein include a device. The device may be used, for example, to perform the methods disclosed in the present application. In some embodiments, the device can be used to enrich for a cell of interest from a cell population. The device may include a substrate, a layer disposed on the substrate, and a cell population dispersed within the layer. The layer (e.g., film 200 depicted in FIG. 2) may include a cytocompatible matrix, and the layer may have a thickness of about 2 mm or less. The cytocompatible matrix can have any of the characteristics described in the present application with regard to the method. For example, the cytocompatible matrix can include a polymer, or can include a hydrogel. The cell population (e.g., cell population 210 depicted in FIG. 2) may have any of the characteristics described in the present application with regard to the method. The substrate (e.g., substrate 220 depicted in FIG. 2) may have any of the characteristics described in the present application with regard to the method.

Some embodiments disclosed herein include a composition. The composition may be used, for example, to perform the methods disclosed in the present application. In some embodiments, the composition can be used to enrich for a cell of interest from a cell population. The composition may include a cytocompatible matrix and one or more labels. The composition may include a cytocompatible matrix, one or more labels, and a cell population. The cytocompatible matrix, the one or more labels, and the cell population can each have any of the characteristics described in the present application with regard to the method.

### EXAMPLES

Additional embodiments are disclosed in further detail in the following examples, which are not in any way intended to limit the scope of the claims.

### Example 1

A 10 mL whole blood sample was obtained from a pregnant subject and anucleate cells were removed using density centrifugation with PERCOLL as the density gradient medium. The blood sample after centrifugation was about 0.15 mL and contained about 0.16 million cells per μL.

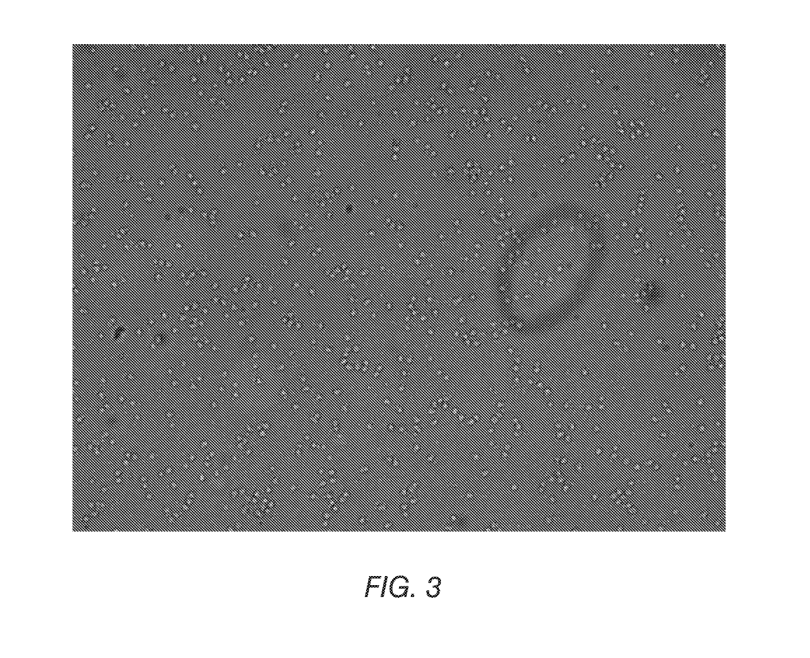

About 0.15 mL of this blood sample was then mixed with about 274 mg of poly(ethylene glycol) diacrylate (PEGDA; 6K Da), about 6.03 mL of 1× PBS, and about 15 mg of a photoinitiator IRGACURE 2959. This mixture was applied to a glass slide to form a film having a thickness of about 40 μm. UV radiation was applied to the film to form a hydrogel film. FIG. 3 shows the cell population dispersed in the hydrogel after photopolymerization using bright field microscopy.

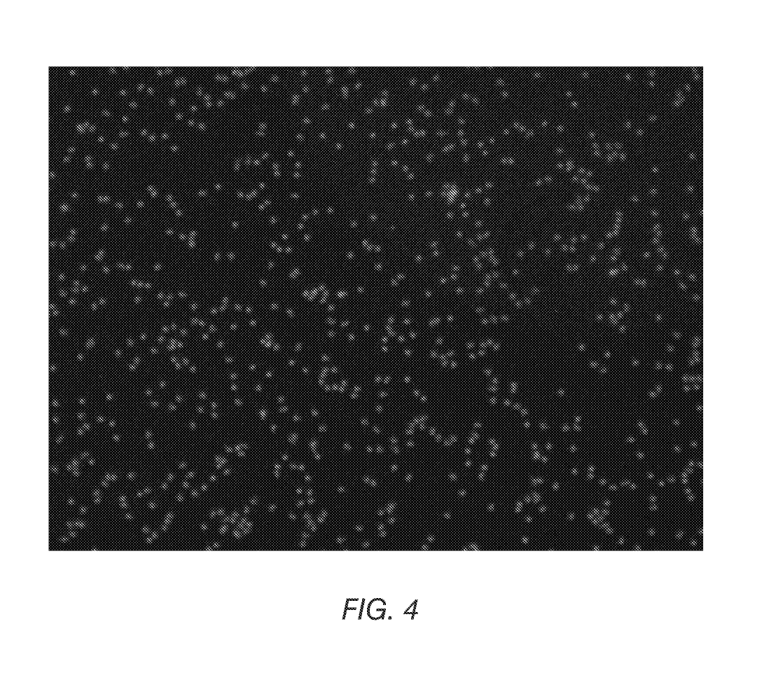

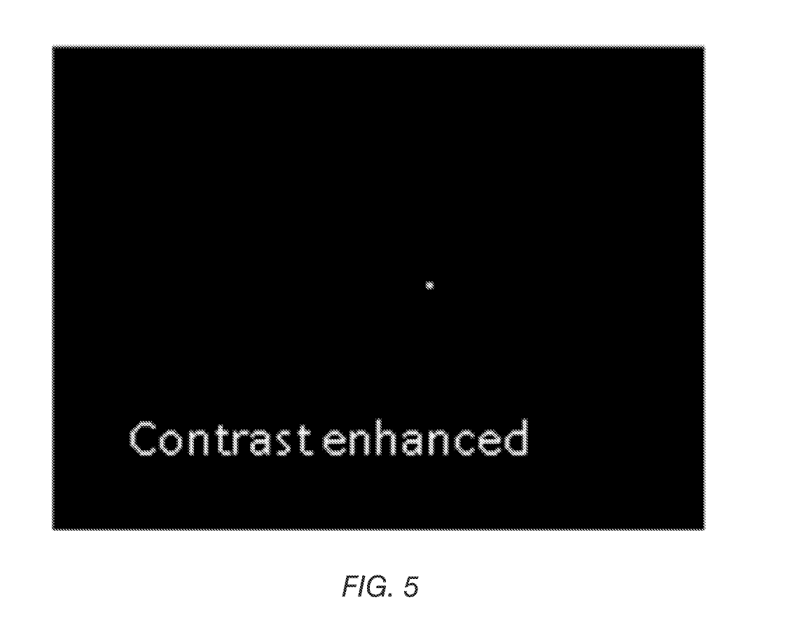

About 5 mL solution containing about 0.02 mg of a fluorescent-tagged, monoclonal antibody for ε-hemoglobin, about 0.05 mg of a fluorescent-tagged, monoclonal antibody for γ-hemoglobin, and about 1 mg of saponin were applied to the surface of the film. The resulting film was viewed using a fluorescence microscope as shown in FIG. 4. FIG. 5 shows a contrast-enhanced image of a target cell identified in a portion of the film using the ε-hemoglobin and γ-hemoglobin antibodies. The identified portions were manually cut and separated from the film.

### Example 2

A blood sample is obtained and centrifuged as described in Example 1. This blood sample is then suspended in a photodegradable hydrogel generally according to the method for hydrogel formation with encapsulated cells disclosed in Kloxin, A. et al., Nature Protocols, Vol. 5(12), pp. 1867-87 (2010), which is incorporated herein by reference in its entirety. The resulting film may have a thickness of about 40 μm.

Fluorescent-tagged, monoclonal antibodies are applied to the film in the same manner as described in Example 1. The resulting film was viewed using a fluorescence microscope and portions of the film exhibiting fluorescence were identified as containing a fetal cell. A mask can be placed over the film with openings corresponding to the location of the identified portions. A 365-nm light source is applied to the film that degrades the identified portions of the film. The degraded portions and the cells within these portions are removed by suction or capillary action.

What is claimed is:

1. A method of enriching for a cell of interest from a heterogeneous cell population, the method comprising:
obtaining a cell population dispersed in a cytocompatible matrix;
applying a label to the cell population to distinguish a cell of interest from one or more cells of the cell population; and
identifying a portion of the cytocompatible matrix containing the cell of interest.

2. The method of claim 1, wherein obtaining the cell population dispersed in the cytocompatible matrix comprises combining the cell population with a composition including a monomer and polymerizing the monomer to form the cytocompatible matrix.

3. The method of any of claims 1 to 2, wherein obtaining the cell population dispersed in a cytocompatible matrix comprises:
combining the cell population with a composition comprising a polymer; and
crosslinking the polymer to form the cytocompatible matrix.

4. The method of claim 3, wherein crosslinking the polymer to form the cytocompatible matrix comprises applying radiation to the polymer, heating the polymer, adjusting a pH of the composition, or combining a crosslinking agent with the composition.

5. The method of any of claims 1 to 4, wherein applying the label to the cell population comprises combining the label with the cell population before the cell population is dispersed in the cytocompatible matrix.

6. The method of any of claims 1 to 4, wherein applying the label to the cell population comprises combining the label with the cell population after crosslinking the polymer.

7. The method of any of claims 1 to 6, wherein applying the label to the cell population comprises applying the label to a surface of the cytocompatible matrix containing the cell population.

8. The method of any of claims 1 to 7, wherein the method comprises applying at least two labels to the cell population.

9. The method of any of claims 1 to 8, wherein a first label and a second label are applied to the cell population, the first label is configured to identify a cellular surface marker, and the second label is configured to identify an intracellular marker or structure.

10. The method of any of claims 1 to 9, wherein identifying a portion of the cytocompatible matrix containing a cell of interest comprises detecting radiation corresponding to at least one of the labels or detecting a magnetic field or a magnetic force corresponding to at least one of the labels.

11. The method of any of claims 1 to 10, further comprising isolating the portion of the cytocompatible matrix containing the cell of interest.

12. The method of claim 11, wherein isolating the portion of the cytocompatible matrix containing the cell of interest comprises mechanically separating the portion of the cytocompatible matrix containing the cell of interest from the cytocompatible matrix.

13. The method of any of claims 11 to 12, wherein isolating the portion of the cytocompatible matrix containing the cell of interest comprises selectively degrading the cytocompatible matrix in the portions of the cytocompatible matrix containing the cell of interest and removing the degraded portions from the cytocompatible matrix.

14. The method of any of claims 11 to 12, wherein isolating the portion of the cytocompatible matrix containing the cell of interest comprises selectively degrading the cytocompatible matrix adjacent to the portion containing the cell of interest and separating the degraded portions from the portion containing the cell of interest.

15. A device for enriching for a cell of interest from a heterogeneous cell population, the device comprising:
a substrate;
a layer disposed on the substrate comprising cytocompatible matrix,
wherein the layer has a thickness of about 2 mm or less; and
a cell population dispersed within the cytocompatible matrix.

16. A composition for enriching for a cell of interest from a heterogeneous cell population, the composition comprising:
a cell population dispersed in a cytocompatible matrix, and one or more labels to distinguish a cell of interest from one or more cells of the cell population.

17. The method, device, or composition, of any of claim 1 or 16, wherein the cytocompatible matrix comprises a polymer.

18. The method, device, or composition, of any of claims 1 to 17, wherein the cytocompatible matrix comprises a hydrogel.

19. The method, device, or composition, of any of claims 1 to 18, wherein the cell population is obtained from a blood sample

20. The method, device, or composition, of claim 19, wherein the blood sample is from a pregnant woman.

21. The method, device, or composition, of any of claims 1 to 20, wherein the cell population comprises maternal cells and fetal cells.

22. The method, device, or composition, of any of claims 1 to 21, wherein the cell of interest is a fetal cell, a cancer cell, or a stem cell.

23. The method, device, or composition, of any of claims 1 to 22, wherein the cytocompatible matrix comprises a polymer selected from the group consisting of poly(alkylene oxide), a starch, a cellulose, a polysaccharide, polyurethane, polyvinyl alcohol, polyvinyl ether, polyacrylate, polyvinylpyrolidone, polyesters, polyacrylamide, polyglycolic acid, polylactic acid, a protein, copolymers thereof and derivatives thereof.

24. The method, device, or composition, of any of claims 1 to 23, wherein the cytocompatible matrix is light-transmissive.

25. The method, device, or composition, of any of claims 1 to 24, wherein the cytocompatible matrix has a light transmittance of at least about 50% for visible light.

26. The method, device, or composition, of any of claims 1 to 25, wherein the cytocompatible matrix has a viscosity of at least about 50 cP.

27. The method, device, or composition, of any of claims 1 to 26, wherein, the cytocompatible matrix is porous.

28. The method, device, or composition, of any of claims 1 to 27, wherein the cytocompatible matrix is photodegradable or enzymatically degradable.

29. The method, device, or composition, of any of claims 1 to 28, wherein the cytocompatible matrix has a mass swelling ratio at approximately equilibrium conditions of less than about 500.

30. The method, device, or composition, of any of claims 1 to 29, wherein the label identifies a cellular surface marker or an intracellular marker or structure.

31. The method, device, or composition, of claim 30, wherein the intracellular marker is a nucleic acid marker or a protein marker.

32. The method, device, or composition, of any of claims 1 to 31, wherein the label is an antibody.

33. The method, device, or composition, of any of claims 1 to 32, wherein the label is a stain, preferably to identify a cellular structure.

34. The method, device, or composition, of any of claims 1 to 33, wherein the stain is a nuclei stain.

35. The method, device, or composition, of claim 34, wherein the stain is hematoxylin, neutral/toluylene red, or Nile blue.

36. The method, device, or composition, of any of claims 1 to 35, wherein at least one label comprises a fluorescent-label, a radio-label, a magnetic particle or a paramagnetic particle.

37. The method, device, or composition, of any of claims 1 to 36, wherein at least one label identifies a marker for a fetal cell.

38. The method, device, or composition, of claim 37, wherein the marker for fetal cell is selected from the group consisting of CD71, CD34, CD45, and CD235a.

39. The method, device, or composition, of any of claims 1 to 38, wherein at least one label identifies a marker for a maternal cell.

40. The method, device, or composition, of claim 39, wherein the marker for the maternal cell is selected from the group consisting of CD2, CD3, CD11b, CD14, CD15, CD16, CD19, CD56, CD123, and CD61.

41. The method, device, or composition, of any of claims 1 to 36, wherein at least one label identifies a marker for a cancer cell.
